(19) United States
(12) Patent Application Publication
(10) Pub. No.: US 2013/0109918 A1
(43) Pub. Date: May 2, 2013

(54) MEDICO-SURGICAL ASSEMBLIES

(71) Applicant: Eric Pagan, Hythe (GB)

(72) Inventor: Eric Pagan, Hythe (GB)

(73) Assignee: SMITHS MEDICAL INTERNATIONAL LIMITED, Ashford (UK)

(21) Appl. No.: 13/809,459

(22) PCT Filed: Jun. 27, 2011
(86) PCT No.: PCT/GB2011/000954

§ 371 Date: Jan. 10, 2013

(30) Foreign Application Priority Data
Jul. 24, 2010 (GB) 1012446.9

(51) Int. Cl. A61M 16/04; A61B 1/06; A61B 1/05; A61B 1/267; A61B 1/00

(52) U.S. Cl. CPC A61M 16/0488; A61B 1/267; A61B 1/00135; A61B 1/05; A61B 1/0676
USPC 600/109; 600/120

(57) ABSTRACT

An endotracheal tube is introduced into the trachea using a video introducer in the form of a bendable rod having a camera and light source at one end and connected to a display monitor via a cable and connector at the other end. The introducer is initially inserted to the trachea carrying a guide sleeve extending along its outside. When correctly inserted, the video introducer is removed by pulling rearwardly to leave the guide sleeve in position. The endotracheal tube is then slid into position along the guide sleeve, following which the guide sleeve is removed to leave the endotracheal tube in position. The guide sleeve could be stiffened after removing the video introducer and before inserting the tube by inserting a stylet or filling the sleeve with a substance.

This invention relates to medico-surgical assemblies of the kind including elongate viewing means arranged to view an anatomical site at its forward end.

The invention is more particularly concerned with medico-surgical assemblies for use in placing apparatus within a patient such as inserting an endotracheal tube into the trachea. Traditional introducers take the form of a simple rod that can be bent to an approximate desired shape and can flex to accommodate the shape of the anatomy during insertion. The introducer may be made with an angled, Coude tip to facilitate introduction. The introducer can be inserted more easily than the tube itself because it has a smaller diameter and can be bent and flex to the ideal shape for insertion. The small diameter also gives the clinician a better view of the trachea around the outside of the introducer. These may be used with or without the aid of a laryngoscope. When the introducer has been correctly inserted, a tube can be slid along the outside of the introducer to the correct location, after which the introducer is pulled out of the tube, which is left in position. Introducers are available from Smiths Medical. GB2312378 describes an introducer moulded of an aliphatic polyurethane material and also describes an earlier introducer made from a braided polyester filament repeatedly coated in layers of resin.

More recently it has been proposed to use fibre optics or a CCD camera with an introducer to provide the clinician with a view of the trachea as the introducer is inserted. WO2007/089491 describes an arrangement with an introducer and a separate camera assembly clipped onto the outside of the introducer, which is removed before an endotracheal tube can be slid along the introducer. Alternative arrangements have a handle at the machine end of the introducer so that the apparatus has to be inserted with the tube already loaded on the introducer from its patient end. Such arrangements are more difficult to use because of the presence of the tube during insertion. WO2010/136748 describes an introducer arrangement with a camera at one end of an introducer and connected via a cable to a display screen at the opposite end. The introducer is disconnected from the display when correctly positioned so that an endotracheal tube can be slid along the introducer into position, following which the introducer is removed by pulling rearwardly from the endotracheal tube. US2007/0112251 describes a fibre-optic endoscope with an outer sheath through the bore of which a catheter can be slid after the endoscope has been removed.

It is an object of the present invention to provide alternative medico-surgical assemblies.

According to one aspect of the present invention there is provided a medico-surgical assembly of the above-specified kind, characterised in that the assembly includes a sleeve slidably mounted on the viewing means and extending from a region adjacent its forward end along a major part of the length of the viewing means, that the viewing means is removable rearwardly from within the sleeve after correct positioning of the viewing means such as to leave the sleeve in position, and that the assembly includes tubular apparatus slidably mounted along the outside of the sleeve after removal of the viewing means such that the tubular apparatus can be slid to the desired position viewed by the viewing means and the sleeve subsequently removed from within the tubular apparatus leaving the tubular apparatus in position.

The tubular apparatus may be an endotracheal tube. The viewing means preferably includes a camera and illumination means at its forward end. The viewing means may include a cable extending from its rear end and terminated by an electrical connector. The sleeve may be arranged such that its stiffness can be selectively increased. The sleeve may have a forward end closed by a transparent window, the sleeve being arranged to be filled with a substance after removal of the viewing means to increase the stiffness of the sleeve. The substance may be selected from a group comprising: liquid, gas, paste, gel, beads, grains or powder. The substance may change properties within the sleeve to increase the stiffness of the sleeve. Alternatively, the sleeve could include a stylet slidable along the sleeve. The sleeve may include a main channel along which the viewing means is received and a minor channel extending along its length, the minor channel being filled with a substance or a stylet after removal of the viewing means, to increase the stiffness of the sleeve.

According to another aspect of the present invention there is provided a technique for introducing a medico-surgical tube into the body including the steps of introducing viewing means into the body under visual monitoring, the viewing means having a sleeve slidably extending along the viewing means, subsequently withdrawing the viewing means but leaving the sleeve in the body in the desired position, then sliding the medico-surgical tube along the outside of the sleeve so that it too is located in the desired position in the body, and subsequently removing the sleeve from within the tube to leave the tube in position.

The method may further include the step of increasing the effective stiffness of the sleeve after removing the viewing means and before sliding the tube along the sleeve.

An endotracheal tube placement assembly according to the present invention will now be described, by way of example, with reference to the accompanying drawings, in which:

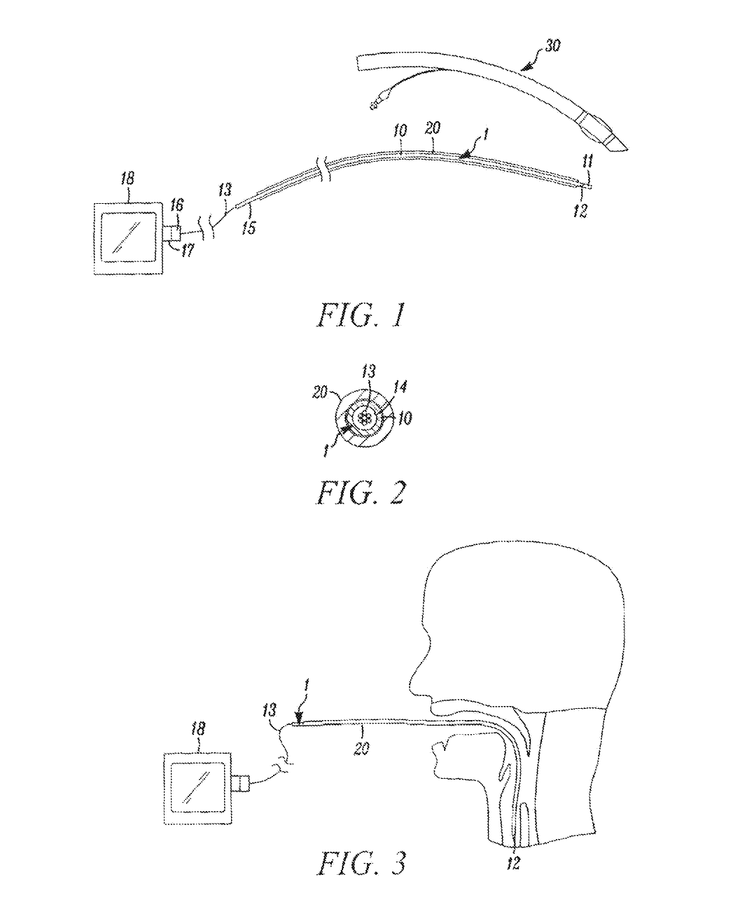

FIG. 1 shows an assembly of viewing means in the form of a video introducer, a guide sleeve and an endotracheal tube;

FIG. 2 is a cross-section view across the video introducer and sleeve;

FIG. 3 illustrates insertion of the video introducer and sleeve;

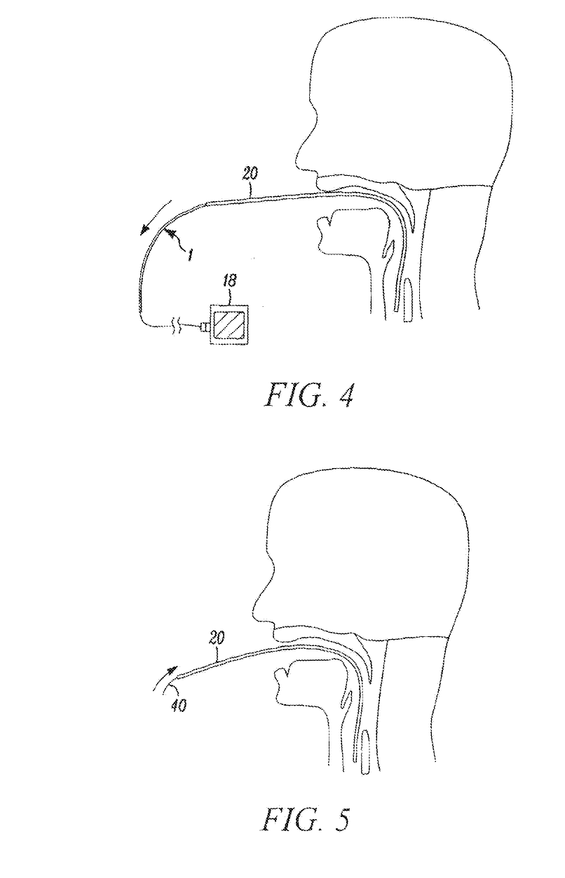

FIG. 4 shows the video introducer being removed and the sleeve left in place;

FIG. 5 shows a subsequent step of effectively stiffening the sleeve by inserting a stylet;

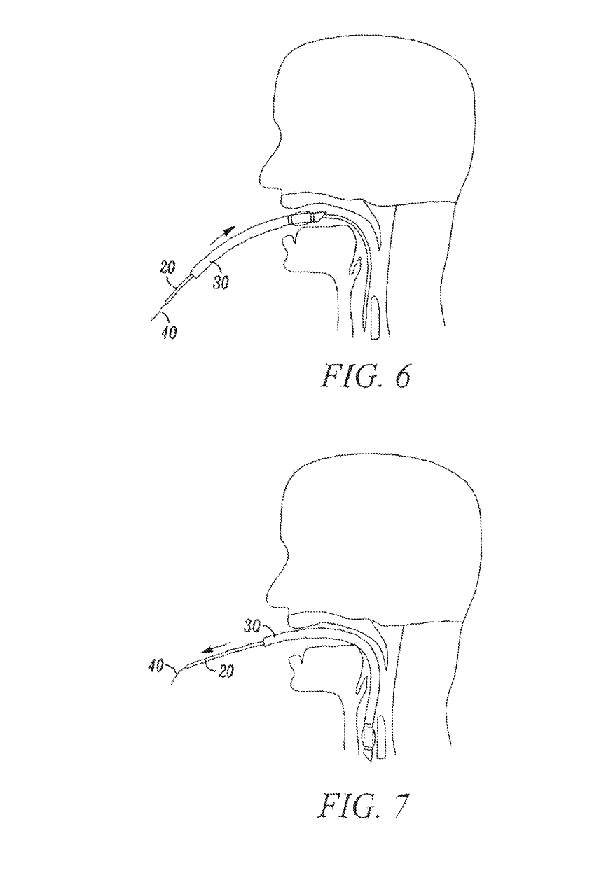

FIG. 6 shows a subsequent step of sliding the endotracheal tube into position along the outside of the sleeve and stylet;

FIG. 7 shows the sleeve and stylet being removed to leave the tube in position;

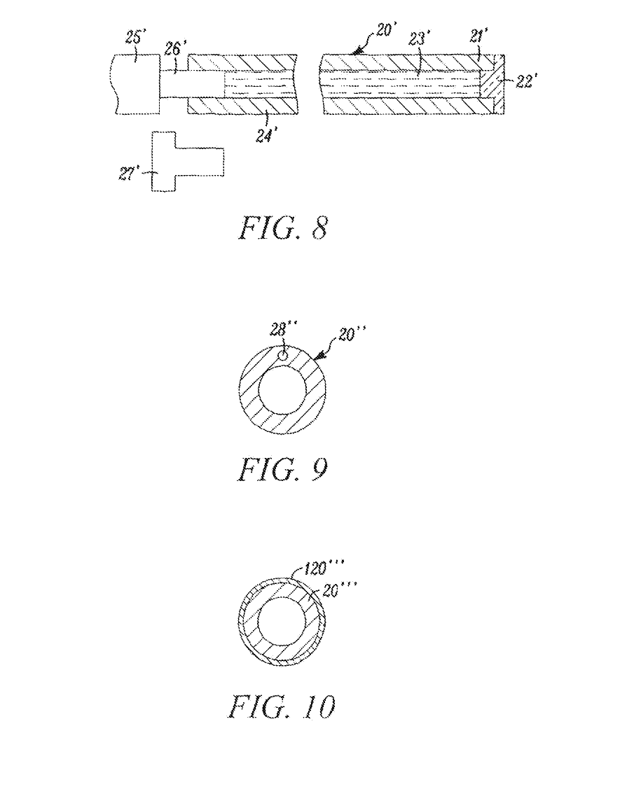

FIG. 8 is an enlarged cross-sectional side elevation of an alternative form of guide sleeve;

FIG. 9 is an enlarged cross-sectional view of another alternative guide sleeve; and

FIG. 10 is an enlarged cross-sectional view of a further alternative guide sleeve.

With reference first to FIGS. 1 and 2, the assembly consists of viewing means or a video introducer 1 in the form of a flexible, bendable rod 10 with a small camera and illumination unit 11 mounted at its forward patient end 12. The video introducer 1 may be substantially as described in WO2010/136748. An electrical cable 13 extends along a bore 14 in the rod 10 to its rear, machine end 15. The cable 13 may extend out of the rod 10 to an electrical connector 16 mated with a cooperating connector 17 on a video display monitor 18. The screen of the video display monitor 18 shows a display representation of the illuminated field of view of the camera unit 11. The video introducer 1 may differ from that described in WO2010/136748 in that there is no need for the connector 16 at the end of the cable 13 to have a low profile since, as will be seen, the endotracheal tube 30 is not introduced along the viewing means itself. The assembly also includes a guide sleeve 20 mounted on the outside of the video introducer 1 as a close sliding fit. The external diameter of the rod 10 can be relatively small, typically around 5-7 mm so the external diameter of the sleeve 20 can be correspondingly small. The sleeve 20 has substantially the same length as the introducer 1 although it could be slightly shorter providing it can extend close to the patient end tip 12 of the introducer at one end and be accessible outside the body at the opposite end. Preferably, the length of the guide sleeve 20 should be such that the length projecting from the patient's mouth when correctly inserted is slightly longer than the length of the endotracheal tube 30. The endotracheal tube 30, is entirely conventional and is separate from the video introducer 1 and guide sleeve 20 before use.

FIG. 3 shows the first step in using the assembly involving inserting the video introducer 1 and sleeve 20 together. Before inserting, the rod 10 is bent by hand to the appropriate shape best suited to the patient's anatomy. The video screen 18 is monitored during insertion to enable the clinician readily to direct the patient end 12 of the rod 10 into the trachea and past the vocal folds into the desired position. The clinician then holds the guide sleeve 20 as he pulls the introducer 1 rearwardly out of the sleeve, leaving the sleeve in position, as shown in FIG. 4. In some cases the guide sleeve 20 may be sufficiently stiff to act by itself as a guide for subsequent insertion of the endotracheal tube 30. However, a relatively stiff guide sleeve could make it more difficult to direct the introducer accurately without trauma. Also, stiffer guide sleeves tend to have a larger external diameter, which might make it more difficult to slide the endotracheal tube along it, especially with smaller diameter tubes, such as paediatric tubes. For these reasons, the guide sleeve may be relatively thin and flimsy with the risk that when the endotracheal tube is slid along it, the guide sleeve could be pulled out of its correct position. In order to prevent this, the present invention contemplates the possible need for some provision to stiffen the guide sleeve 20 after removal of the introducer 1. FIG. 5 shows one simple way of doing this, namely by inserting a stylet 40 along the sleeve 20 after having removed the video introducer 1 so that the endotracheal tube 30 is slid into position in the manner shown in FIG. 6, along the combination of the guide sleeve and stylet. When the tube 30 has been fully inserted, the guide sleeve 20 and stylet 40 are pulled rearwardly out of the rear end of the tube in the manner shown in FIG. 7, leaving the tube in position. The stylet 40 is preferably appropriately marked to indicate how far it should be inserted along the guide sleeve 20; and the guide sleeve is preferably marked to indicate how far the endotracheal tube 30 should be inserted along the guide sleeve.

The arrangement described above enables the video introducer 1 to be positioned without the presence of the endotracheal tube 30 itself, thereby facilitating manipulation of the introducer. Because the endotracheal tube 30 is slid along the guide sleeve 20 and not along the video introducer 1 itself there is no need for the connector 16 to be small enough to pass along the bore of the endotracheal tube. This is an advantage, especially in smaller gauge tubes. The sleeve does not need to be wide enough to receive a tube within it so does not need to be much wider than the viewing means. In this way, the sleeve does not create a significant obstacle to viewing the insertion of the tube should the clinician wish to do this.

The guide sleeve 20 could be stiffened in other ways apart from the stylet 40 inserted along its bore.

FIG. 8 shows an alternative guide sleeve 20′, which is closed at its forward patient end 21′ by a transparent cap 22′. The cap 22′ enables the video introducer 1 to view the patient's anatomy in the trachea through the cap when inserted in the guide sleeve 20′. The guide sleeve 20′ is stiffened after removal of the video introducer 1 by filling the sleeve with a substance 23′ such as a liquid, gas, paste, gel, beads, grains or the like, hence the need for the cap 22′. The filling 23′ could be of a substance that changes properties within the sleeve 20′, such as by stiffening or hardening. Alternatively, the bore along the sleeve 20′ could be evacuated by applying a vacuum; the internal profile of the sleeve could be modified such as with webs or the like so that it collapses and supports itself. The substance or vacuum is applied to the rear, machine end 24′ of the sleeve 20′ such as by means of a syringe 25′, the nose 26′ of which is inserted in the bore of the sleeve. When the sleeve 20′ has been filled, the syringe 25′ is removed and the sleeve is closed at its rear-end by a plug 27′.The presence of the filling 23′ in the sleeve 20′ makes the sleeve slightly stiffer and makes it less likely to collapse and buckle on flexing. This reduces the risk that the guide sleeve 20′ will become dislodged from its desired position while the endotracheal tube 30 is being threaded along it.

FIG. 9 shows another alternative guide sleeve 20″ having a minor channel or lumen 28″ extruded within the wall of the tube in addition to the main channel 29″ within which the viewing means is received. The lumen 28″ is filled or evacuated or is used to receive a stylet in a similar manner to the sleeve shown in FIG. 8. The lumen 28″ could be of any shape and could be a complete circumferential gap dividing the sleeve into inner and outer layers.

FIG. 10 shows a further alternative guide sleeve 20″' having an outer layer 120″' that can be activated to make it more rigid. In particular, the outer layer 120″ could be of an electroconductive polymer. This could be arranged to cooperate with a suitable material incorporated within the sleeve.

It will be appreciated that the invention has application wherever medico-surgical apparatus needs to be placed within a patient and is not limited to tracheal applications.

1-12. (canceled)

13. A medico-surgical assembly including an elongate viewing device arranged to view an anatomical site at its forward end and to provide an output at its rear end representative of an image of the anatomical site, characterised in that the assembly includes a sleeve slidably mounted on the outside of the viewing device and extending from a region adjacent its forward end along a major part of the length of the viewing device that the viewing device is removable rearwardly from within the sleeve after correct positioning of the viewing device such as to leave the sleeve in position, and that the assembly includes tubular apparatus slidably mounted along the outside of the sleeve after removal of the viewing device such that the tubular apparatus can be slid to the desired position viewed by the viewing means and the sleeve subsequently removed from within the tubular apparatus leaving the tubular apparatus in position.

14. An assembly according to claim 13, characterised in that the tubular apparatus is an endotracheal tube

15. An assembly according to claim 13, characterised in that the viewing device means includes a camera and a light source at its forward end.

16. An assembly according to claim 15, characterised in that the viewing device includes a cable extending from its rear end and terminated by an electrical connector.

17. An assembly according to claim 13, characterised in that the sleeve is arranged such that its stiffness can be selectively increased.

18. An assembly according to claim 17, characterised in that the sleeve has a forward end that is closed by a transparent window and that the sleeve is arranged to be filled with a substance after removal of the viewing device to increase the stiffness of the sleeve.

19. An assembly according to claim 18, characterised in that the substance is selected from a group comprising: liquid, gas, paste, gel, beads, grains and powder.

20. An assembly according to claim 18, characterised in that the substance changes properties within the sleeve to increase the stiffness of the sleeve.

21. An assembly according to claim 13, characterised in that the sleeve includes a stylet slidable along the sleeve.

22. An assembly according to claim 13, characterised in that the sleeve includes a main channel along which the viewing device is received and a minor channel extending along its length, and that the minor channel is filled with a substance or a stylet after removal of the viewing device to increase the stiffness of the sleeve.

23. A technique for introducing a medico-surgical tube into the body including the steps of introducing an assembly including a viewing device into the body under visual monitoring from an output of the viewing device, the assembly having a sleeve slidably extending along the outside of the viewing device, subsequently withdrawing the viewing device but leaving the sleeve in the body in the desired position, then sliding the medico-surgical tube along the outside of the sleeve so that it too is located in the desired position in the body, and subsequently removing the sleeve from within the tube to leave the tube in position.

24. A technique according to claim 11, including the step of increasing the effective stiffness of the sleeve after removing the viewing device and before sliding the tube along the sleeve.
